(19) United States
(12) Patent Application Publication
(10) Pub. No.: US 2026/0140576 A1
(43) Pub. Date: May 21, 2026

(54) STERILE STYLUS

(71) Applicant: Boston Scientific Scimed, Inc., Maple Grove, MN (US)

(72) Inventors: Judith Tiferes Wang, Shoreview, MN (US); Kristi Mae Flury, Maple Grove, MN (US); Alan Torborg, Minneapolis, MN (US)

(73) Assignee: Boston Scientific Scimed, Inc., Maple Grove, MN (US)

(21) Appl. No.: 19/395,562

(22) Filed: Nov. 20, 2025

Related U.S. Application Data
(60) Provisional application No. 63/722,826, filed on Nov. 20, 2024.

(51) Int. Cl. G06F 3/0354; A61B 50/30; G06F 3/039

(52) U.S. Cl. CPC G06F 3/03545; A61B 50/30; G06F 3/0393

(57) ABSTRACT

Medical device kits and methods for using medical devices are disclosed. An example medical device kit may include a sterile package. A stylus may be disposed within the sterile package. The stylus may be configured to interact with a touchscreen interface.

### CROSS-REFERENCE TO RELATED APPLICATIONS

This application claims the benefit of priority under 35 U.S.C. §119 of U.S. Provisional Application No. 63/722,826, filed November 20, 2024, the entire disclosure of which is hereby incorporated by reference.

### TECHNICAL FIELD

The present disclosure pertains to medical devices, and methods for manufacturing medical devices. More particularly, the present disclosure pertains to a sterile stylus.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A medical device is disclosed. The medical device comprises: a sterile package; and a stylus disposed within the sterile package, the stylus being configured to interact with a touchscreen interface of a computing device.

Alternatively or additionally to any of the embodiments above, further comprising a second sterile package and a medical device or accessory disposed within the second sterile package.

Alternatively or additionally to any of the embodiments above, the medical device or accessory includes a portable computing device.

Alternatively or additionally to any of the embodiments above, the portable computing device includes a touchscreen tablet.

Alternatively or additionally to any of the embodiments above, the medical device or accessory includes a catheter.

Alternatively or additionally to any of the embodiments above, the medical device or accessory includes an imaging catheter.

Alternatively or additionally to any of the embodiments above, the sterile package and the second sterile package are separated from one another.

Alternatively or additionally to any of the embodiments above, further comprising an outer package containing the sterile package and the second sterile package.

Alternatively or additionally to any of the embodiments above, the stylus is configured to be removed from the sterile package.

Alternatively or additionally to any of the embodiments above, the stylus is configured to be removed from the sterile package within a sterile field of a clinical setting.

A medical device kit is disclosed. The kit comprises: a first sterile package; a portable computing device disposed within the first sterile package, the portable computing device having a touchscreen interface; a second sterile package; and a stylus disposed within the second sterile package, the stylus being configured to interact with the touchscreen interface.

Alternatively or additionally to any of the embodiments above, the first sterile package includes a sleeve disposed on the portable computing device.

Alternatively or additionally to any of the embodiments above, the portable computing device includes a touchscreen tablet.

Alternatively or additionally to any of the embodiments above, the stylus is configured to be removed from the second sterile package.

Alternatively or additionally to any of the embodiments above, the stylus is configured to be removed from the second sterile package within a sterile field of a clinical setting.

Alternatively or additionally to any of the embodiments above, the first sterile package and the second sterile package are separated from one another.

Alternatively or additionally to any of the embodiments above, further comprising an outer package containing the first sterile package and the second sterile package.

A method for using a touchscreen tablet within a sterile field of a clinical setting is disclosed. The method comprises: disposing the touchscreen tablet within a first sterilized package; disposing the first sterilized package within the sterile field of the clinical setting; disposing a second sterilized package having a sterile stylus disposed therein within the sterile field; removing the sterile stylus from the second sterilized package; and engaging the sterile stylus with the touchscreen tablet.

Alternatively or additionally to any of the embodiments above, the first sterilized package and the second sterilized package are disposed within an outer package.

Alternatively or additionally to any of the embodiments above, the touchscreen tablet has a touchscreen interface, and wherein engaging the sterile stylus with the touchscreen tablet includes engaging the sterile stylus with the touchscreen interface of the touchscreen tablet.

A medical device kit is disclosed. The kit comprises: a first sterile package; a medical device or accessory disposed within the first sterile package; a second sterile package; and a stylus disposed within the second sterile package, the stylus being configured to interact with a touchscreen interface.

Alternatively or additionally to any of the embodiments above, the medical device or accessory includes a portable computing device.

Alternatively or additionally to any of the embodiments above, the portable computing device includes a touchscreen tablet.

Alternatively or additionally to any of the embodiments above, the first sterile package includes a sleeve disposed on the portable computing device.

Alternatively or additionally to any of the embodiments above, the medical device or accessory includes a catheter.

Alternatively or additionally to any of the embodiments above, the medical device or accessory includes an imaging catheter.

Alternatively or additionally to any of the embodiments above, the stylus is configured to be removed from the second sterile package.

Alternatively or additionally to any of the embodiments above, the stylus is configured to be removed from the second sterile package within a sterile field of a clinical setting.

Alternatively or additionally to any of the embodiments above, the first sterile package and the second sterile package are separated from one another.

Alternatively or additionally to any of the embodiments above, further comprising an outer package containing the first sterile package and the second sterile package.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically depicts an example clinical setting.

FIG. 2 schematically depicts an example clinical setting.

FIG. 3 schematically depicts an example clinical setting.

FIG. 4 schematically depict an example medical device kit.

FIG. 5 schematically depict an example medical device kit.

FIG. 6 schematically depict an example medical device kit.

FIG. 7 schematically depict an example medical device kit.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

In clinical/hospital settings, a variety of medical devices may be utilized as well as companion devices and/or accessories. Some devices or accessories may be intended for use within a sterile field, whereas other devices may remain outside the sterile field. One example companion device or accessory may be a touchscreen device or tablet. Such a tablet may run software that interacts with or controls other medical devices, displays data or images captured by other medical devices, etc. For example, the AVVIGO™+ Multi-Modality Guidance System, commercially available from Boston Scientific, utilizes a tablet interface in conjunction with a pressure wire (e.g., the Comet™ II Pressure Wire, commercially available from Boston Scientific) or intravascular ultrasound (IVUS) catheter (e.g., the OPTICROSS™ Imaging Catheter, commercially available from Boston Scientific). The AVVIGO™+ System includes a touchscreen tablet interface. When the tablet is within the sterile field, a clinician may interact with the tablet (e.g., where the tablet may be enclosed within a sterile package or sleeve) using gloved hands or the like. Disclosed herein are kits and/or accessories that are configured to be used in within the sterile field. Such devices may include a sterile stylus for use within the sterile field.

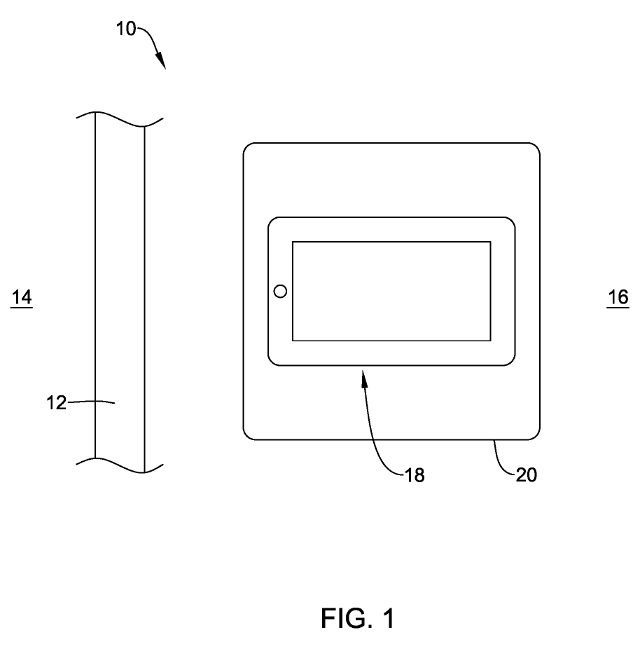

FIG. 1 schematically illustrates an example clinical setting, generally denoted by reference number 10, which may include a barrier 12 that separates the clinical setting 10 between a non-sterile field 14 and a sterile field 16. In this example, a portable computing device 18 is shown disposed within the sterile field 16. The form of the portable computing device 18 may vary. In at least some instances, the portable computing device 18 may include a tablet, for example having a touchscreen tablet having a touchscreen interface. It can be appreciated that electronic devices such as the portable computing device 18 are generally not well suited for sterilization (e.g., steam/autoclave sterilization, etc.). In order to allow for repeated uses of the portable computing device 18 within the sterile field 16, the portable computing device 18 may be disposed within a sterile package or drape 20 (e.g., a clear drape). The sterile package 20 can be disposed over the portable computing device 18, which may create a sterile field for the computing device 18 and/or allow the portable computing device 18 to enter a sterile field 16. Once within the sterile field 16, the portable computing device 18 may be used in conjunction with a clinical intervention. When the clinical intervention is completed, the portable computing device 18 may be removed from the sterile field 16 and the sterile package 20, and the process can be repeated for subsequent interventions.

Interacting the with the portable computing device 18 via a touchscreen interface may include a clinician touching the screen of the portable computing device 18 with a gloved hand. Because gloves may interfere with resistive, electrostatic, or capacitive interaction between the hands of a clinician and the touchscreen interface of the portable computing device 18, it may be challenging to reliably interface with the portable computing device 18. It may be desirable to use a suitable interface device or stylus in order to reliable intact with a resistive, electrostatic, or capacitive touchscreen.

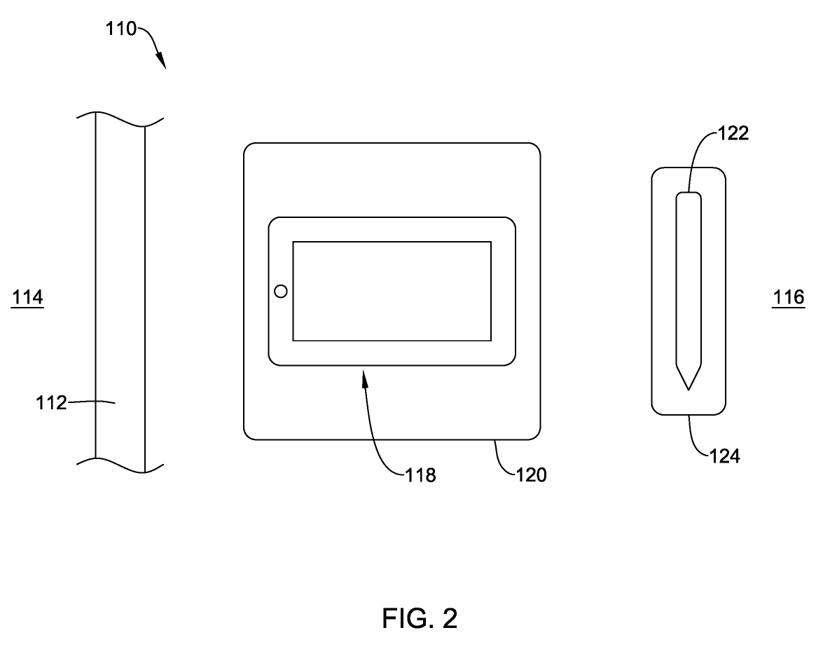

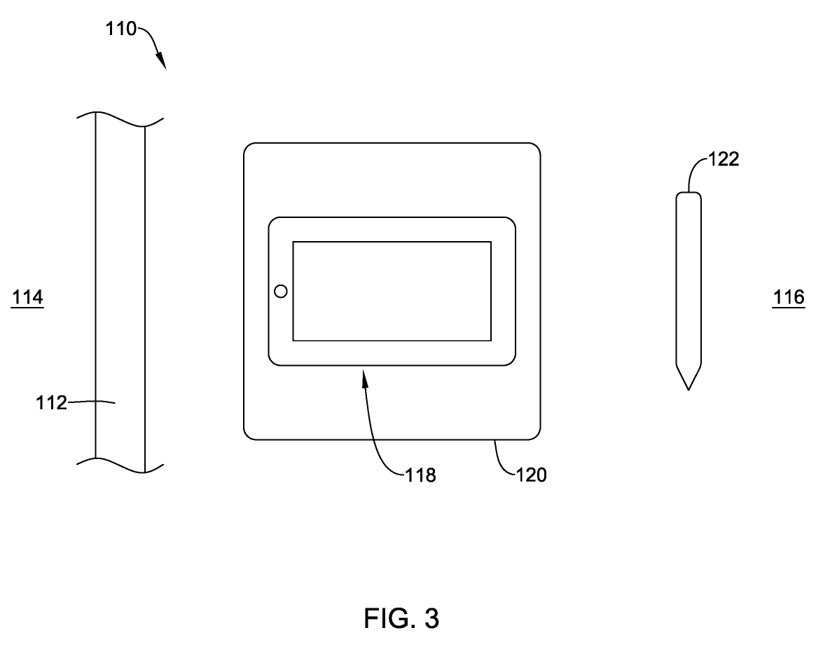

FIG. 2 schematically illustrate another example clinical setting 110 that includes a barrier 112 separating the clinical setting 110 between a non-sterile field 114 and a sterile field 116. In this example, a portable computing device 118 (e.g., which may be similar in form and function to other portable computing devices disclosed herein) is shown disposed within the sterile field 116. The portable computing device 118 may be disposed within a sterile package or sleeve 120. An input device such as a stylus 122 may also be disposed within the sterile field 116. The stylus 122 may be disposed within another (e.g., second) sterile package or sleeve 124. The stylus 122 may be a resistive, electrostatic, capacitive, or similar type of device, suitable for interacting with the touchscreen interface of the portable computing device 118. The stylus 122 may be sterile within the sterile/sterilized package 124 so that the stylus 122 can be removed from the sterilized package 124 while within the sterile field 116 for use within the sterile field 116. For example, FIG. 3 depicts that the stylus 122 removed from the sterile package 124 for use interfacing with the portable computing device 118, for example within the sterile field 116. If desired, the stylus 122 may be re-sterilized and/or put into another sterilized package for reuse.

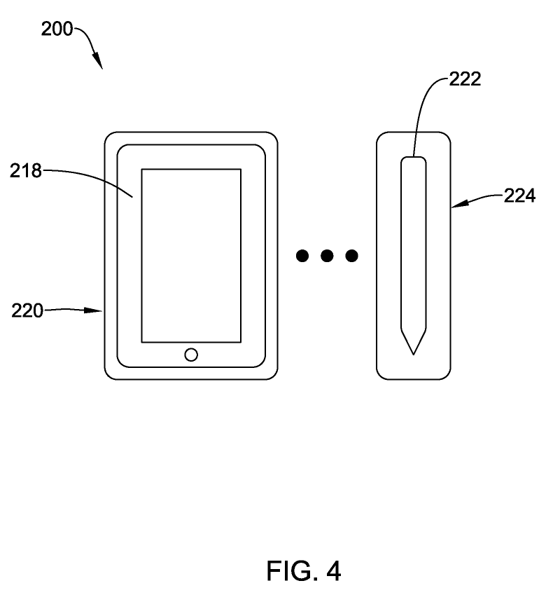

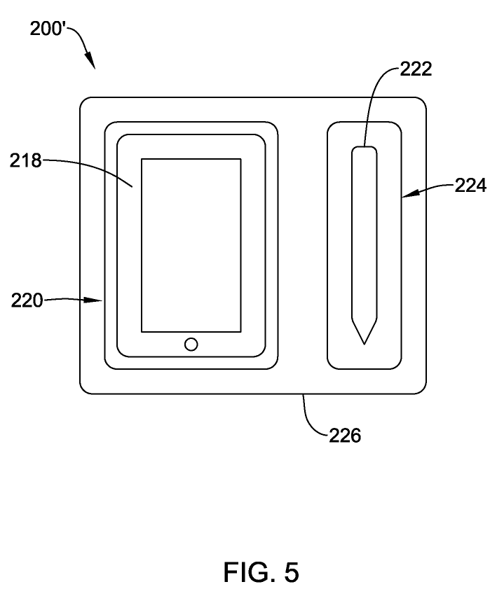

Different combinations of devices or kits are contemplated that may include a sterile or sterilizable stylus for use within a clinical setting. For example, FIG. 4 illustrates one example kit 200. The kit 200 may include a first sterile package 220 having a device such a portable computing device 218 disposed therein. The kit 200 may also include a second sterile package 224 having a stylus 222 disposed therein. In some instances, the first sterile package 220 and the second sterile package 224 may be separated or separable from one another. In other words, the first sterile package 220 and the second sterile package 224 may not be in a singular, common outer package other than, in some examples, a shipping box or container that may include multiple devices/packages. In other instances, the first sterile package 220 and the second sterile package 224 may be disposed together within an outer package 226, forming a kit 200’ as shown in FIG. 5.

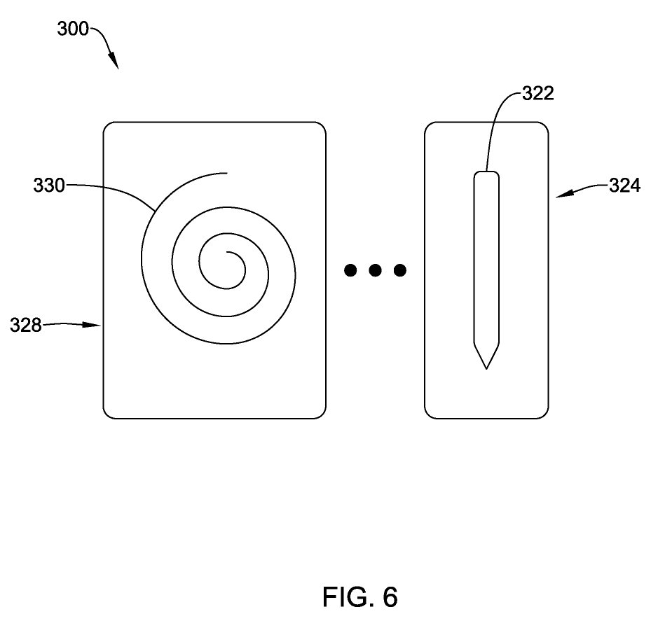

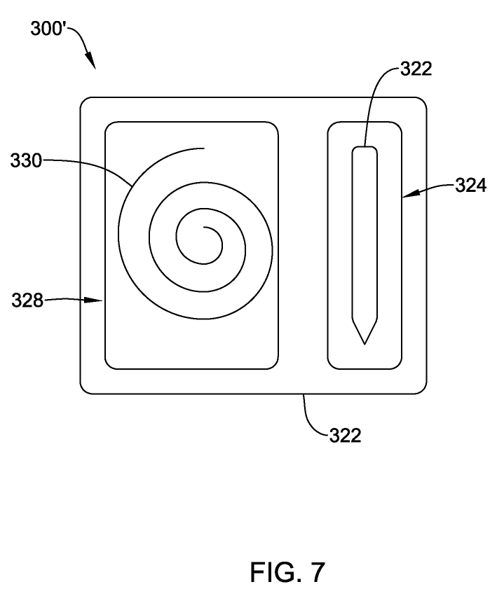

While kits that combine a portable computing device and sterilize stylus are contemplated, other kits are contemplated that include different groupings/arrangements of devices. For example, FIG. 6 illustrates another example kit 300. The kit 300 may include a first sterile package 328 having a device such a catheter 330 disposed therein. The catheter 330 may any one of a variety of different types of catheters/devices such as guide catheters, stent delivery systems, imaging catheters (e.g., IVUS imaging catheters), etc. The kit 300 may also include a second sterile package 324 having a stylus 322 disposed therein. In some instances, the first sterile package 328 and the second sterile package 324 may be separated or separable from one another. In other instances, the first sterile package 320 and the second sterile package 324 may be disposed together within an outer package 332, forming a kit 300’ as shown in FIG. 7.

Other kits are contemplated. One example kit may simply include a sterile stylus disposed within a sterilized/sterilizable package. This may resemble the stylus 222 and sterile package 224 depicted in FIGS. 4-5. Another example kit may include a portable computing device disposed within a sterilized/sterilizable package, a catheter or medical device disposed within a (second) sterilized/sterilizable package, and a sterile stylus disposed within a (third) sterilized/sterilizable package. The portable computing device disposed within a sterilized/sterilizable package may resemble the portable computing device 218 and sterile package 220 shown in FIGS. 4-5. The catheter or medical device disposed within a (second) sterilized/sterilizable package may resemble the medical device 330 and sterile package 328 shown in FIGS. 6-7. The sterile stylus disposed within a (third) sterilized/sterilizable package may resemble the stylus 222 and sterile package 224 depicted in FIGS. 4-5. In some instances, the three sterilized/sterilizable packages may be all disposed within an outer sterilized/sterilizable package.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A medical device, comprising:
a sterile package; and
a stylus disposed within the sterile package, the stylus being configured to interact with a touchscreen interface of a computing device.

2. The medical device of claim 1, further comprising a second sterile package and a medical device or accessory disposed within the second sterile package.

3. The medical device of claim 2, wherein the medical device or accessory includes a portable computing device.

4. The medical device of claim 3, wherein the portable computing device includes a touchscreen tablet.

5. The medical device of claim 2, wherein the medical device or accessory includes a catheter.

6. The medical device of claim 2, wherein the medical device or accessory includes an imaging catheter.

7. The medical device of claim 2, wherein the sterile package and the second sterile package are separated from one another.

8. The medical device of claim 7, further comprising an outer package containing the sterile package and the second sterile package.

9. The medical device of claim 1, wherein the stylus is configured to be removed from the sterile package.

10. The medical device of claim 1, wherein the stylus is configured to be removed from the sterile package within a sterile field of a clinical setting.

11. A medical device kit, the kit comprising:
a first sterile package;
a portable computing device disposed within the first sterile package, the portable computing device having a touchscreen interface;
a second sterile package; and
a stylus disposed within the second sterile package, the stylus being configured to interact with the touchscreen interface.

12. The kit of claim 11, wherein the first sterile package includes a sleeve disposed on the portable computing device.

13. The kit of claim 11, wherein the portable computing device includes a touchscreen tablet.

14. The kit of claim 11, wherein the stylus is configured to be removed from the second sterile package.

15. The kit of claim 11, wherein the stylus is configured to be removed from the second sterile package within a sterile field of a clinical setting.

16. The kit of claim 11, wherein the first sterile package and the second sterile package are separated from one another.

17. The kit of claim 11, further comprising an outer package containing the first sterile package and the second sterile package.

18. A method for using a touchscreen tablet within a sterile field of a clinical setting, the method comprising:
disposing the touchscreen tablet within a first sterilized package;
disposing the first sterilized package within the sterile field of the clinical setting;
disposing a second sterilized package having a sterile stylus disposed therein within the sterile field;
removing the sterile stylus from the second sterilized package; and
engaging the sterile stylus with the touchscreen tablet.

19. The method of claim 18, wherein the first sterilized package and the second sterilized package are disposed within an outer package.

20. The method of claim 18, wherein the touchscreen tablet has a touchscreen interface, and wherein engaging the sterile stylus with the touchscreen tablet includes engaging the sterile stylus with the touchscreen interface of the touchscreen tablet.
